(19) United States
(12) Patent Application Publication
(10) Pub. No.: US 2003/0064437 A1
(43) Pub. Date: Apr. 3, 2003

(54) Expression system for recombinant proteins

(76) Inventors: Nick Wan, Auburndale, MA (US); Henry Hoppe IV, Acton, MA (US); Jason C. Goodrick, San Francisco, CA (US); Bernhard M. Schilling, Syracuse, NY (US)

Correspondence Address: Jennifer A. Tegfeldt, Esq., Genzyme Corporation, Legal Department, One Kendall Square, Cambridge, MA, 02139, US

(21) Appl. No.: 10/045,507

(22) Filed: Nov. 7, 2001

Related U.S. Application Data
(60) Provisional application No. 60/248,806, filed on Nov. 15, 2000.

(51) Int. Cl. C12P 21/02; C07H 21/04; C12N 9/16; C12N 9/24; C12N 9/64; C12N 1/18; C12N 15/74

(52) U.S. Cl. 435/69.1; 435/200; 435/226; 435/196; 435/254.23; 435/320.1; 536/23.2

(57) ABSTRACT

A continuous fermentation process has been developed in Pichia pastoris (P. pastoris) in order to produce large quantities of recombinant human proteins. High expression levels have been demonstrated in continuous production of the enzyme by P. pastoris with a constitutive promoter in a 1.5-liter working volume fermenter using either glucose or glycerol as the carbon source.

The fermentation could be extended for long periods of time with a excellent steady-state protein concentration and cell densities achieved. No proteolytic degradation of the enzyme was seen in the continuous fermentation mode.

### BACKGROUND OF THE INVENTION

Pichia Pastoris Expression Systems

P. pastoris was recognized in the seventies as a potential source for production of single-cell proteins for feed supplements due to its rather unique ability to anabolize methanol to very high cell mass. Expression of recombinant proteins in P. pastoris has been in development since the late 1980's and the number of recombinant proteins produced in P. pastoris have increased significantly in the past several years (Cregg, et al., 1993; Sberna, et al., 1996). P. pastoris is a desirable expression system because it grows to extremely high cell densities in very simple and defined media free of animal-derived contaminants. The defined growth medium used for the cultivation of P. pastoris is inexpensive and free of toxins or pyrogens. Furthermore, the yeast itself does not present problems in terms of endotoxin production or viral contamination.

Additionally Pichia can secrete expressed proteins at very high levels (>1 g/L and up to 80% of total cellular protein for some proteins) (Sberna, et al 1996). Unlike bacteria, it is capable of producing complex proteins with post-translational modifications, e.g., correct folding, glycosylation, and proteolytic maturation (White, et al. 1994; Sberna, et al. 1996). Pichia are different than Saccharomyces in that they do not tend to hyperglycosylate proteins (oligosaccharide chains of 8-14 mannose) (Grinna & Tschopp 1989) and the highly immunogenic α1,3-mannose structure is not found (Cregg et al., 1993). Pichia generally secretes the expressed proteins into the medium in a fairly pure form (30-80% of total secreted proteins) (Sberna, et al.) thus allowing for easy purification. It is also capable of growing in a very wide pH range, from 3 to 7.

Traditionally, P. pastoris fermentations are performed in batch/fed-batch modes using a methanol inducible system, Chen et al. (1). Some researchers have adapted this system to continuous or continuous perfusion fermentation with limited success (Brierley et al.; Chen et al. (2); Cregg; Digan et al., 1989). Recently, constitutive promoters (e.g., Glyceraldehyde-3-phosphate Dehydrogenase, GAP) have been developed for the P. pastoris expression system (Waterham et al., 1997). These vectors allow for continuous production of the desired recombinant protein without methanol induction and are now readily available commercially (Invitrogen, San Diego, Calif.). This system is more desirable for large scale productions because the hazard and cost associated with large volumes of methanol are eliminated. Using constitutive constructs, glucose can be chosen as an inexpensive and efficient carbon source. P. pastoris high yield expression systems have been successfully utilized to produce large quantities of biologically active, highly disulfide-bonded recombinant proteins of commercial interest e.g. IGF-1, HSA, TNF, Human Interleukin-2. (Buckholz et al (1991)., Cregg et al. (1993); Ohtani et al. (1998); White et al.(1994)).

EntreMed, Inc. was recently reported to have successfully used the P. pastoris expression system for the production of the proteins Angiostatin® and Endostatin™ (Wells (1998)). Proteins produced by P. pastoris are usually folded correctly and secreted into the medium, facilitating the subsequent downstream processing. P. pastoris has further been proven to be capable of N- and O-linked glycosylation and other post-translational protein modifications similar to that found in mammalian cells (Buckholtz et al; Cregg et al (1995); Cregg et al. (1993)).

The continuous production mode offers, in comparison to fed-batch fermentation, advantages in terms of higher volumetric productivity, product quality, and product uniformity as the exposure of the product to proteolytic enzymes, the possibility of protein aggregation, oxidation or inactivation is significantly reduced. A continuous production process for rh-Chitinase using a constitutive P. pastoris expression system was recently developed by the inventors and compared very favorably in terms of cost effectiveness, development time, and effort to expression of rh-Chitinase in mouse C127 cells.

One major drawback of the P. pastoris system is the degradation of the secreted protein by its own proteases (Boehm 1999). Degradation is increased when high-density fermentation is employed since the concentration of proteases in the fermentation broth also increases. Several strategies have been tried including the addition of an amino acid-rich supplement, changing of growth pH (3-7), and use of a protease-deficient host, but they have only worked with limited success. Another potential disadvantage of P. pastoris compared to mammalian cell expression systems is hyperglycosylation, which may cause differences in immunogenicity, specific activity, and serum half life of the recombinant protein.

Lysosomal Storage Diseases

Several of the over thirty known lysosomal storage diseases (LSDs) are characterized by a similar pathogenesis, namely, a compromised lysosomal hydrolase. Generally, the activity of a single lysosomal hydrolytic enzyme is reduced or lacking altogether, usually due to inheritance of an autosomal recessive mutation. As a consequence, the substrate of the compromised enzyme accumulates undigested in lysosomes, producing severe disruption of cellular architecture and various disease manifestations.

Gaucher's disease is the oldest and most common lysosomal storage disease known. Type 1 is the most common among three recognized clinical types and follows a chronic course which does not involve the central nervous system (“CNS”). Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazim (see generally Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W. B. Saunders Co., pages 1461-1507). Also known as glucosylceramide lipidosis, Gaucher's disease is caused by inactivation of the enzyme glucocerebrosidase and accumulation of glucocerebroside. Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme sphingomyelinase in Types A and B disease, with 27-fold more residual enzyme activity in Type B (see Kolodny et al., 1998, Id.). The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. CNS involvement differentiates the major types of Niemann-Pick. While most Type B patients do not experience CNS involvement, it is characteristic in Type A patients. The kidneys are only moderately involved in Niemann Pick disease.

### SUMMARY OF THE INVENTION

Accordingly, the present invention provides methods for the production of recombinant proteins, such as glucocerebrosidase, sphingomyelinase and others, with high-mannose carbohydrate structure. The methods comprise culturing cells of Pichia pastoris which cells have been recombinantly engineered to comprise a DNA molecule which encodes the protein of interest, such as glucocerebrosidase or sphingomyelinase, under conditions suitable for the expression of said DNA molecule. The methods of the present invention are particularly applicable for production of proteins intended to be targeted to macrophages, including Kupffer cells. The methods of the invention may also be useful for targeting other cells which contain surface mannose receptors.

The methods are preferably performed under conditions suitable for continuous fermentation of Pichia pastoris. The DNA molecules for use in the present invention preferably comprise a constitutive promoter operatively linked to the coding sequence of interest. One particularly well-suited constitutive promoter is the GAPDH promoter from yeast. In other embodiments, the present invention also provides methods for the purification of recombinant proteins, such as recombinant human glucocerebrosidase or recombinant human sphingomyelinase, with high-mannose carbohydrate structure. The method preferably comprises culturing cells of Pichia pastoris, which cells comprise a DNA molecule which encodes the protein of interest, such as glucocerebrosidase or sphingomyelinase, under conditions suitable for the expression of said DNA molecule to produce recombinant protein in a cell culture, and purifying said produce purified recombinant human protein from the cell culture. The purification can be accomplished by any suitable conventional means for isolating protein from other components of cell cultures, including HPLC, affinity columns, column chromatography, gel chromatography.

One important advantage of the present methods is that, because Pichia produces proteins with high-mannose glycosylation, fewer modification steps will be needed in order to remove other complex carbohydrates from the recombinant protein in order to expose high-mannose moieties. This will simplify the production of recombinant protein, if a high-mannose glycosylation product is desired. Such a product may be desirable, for example, if targeting of the recombinantly produced protein to macrophages is desired, such as with certain of the lysosomal storage enzymes, including glucocerebrosidase and sphingomyelinase.

The present invention provides methods for continuous high cell density fermentation system for the production of recombinant human proteins, including Chitinase, glucocerebrosidase, sphingomyelinase and others, preferably using constitutive promoters, such as the GAPDH promoter, in which proteolytic degradation of the product was reduced or even undetectable. Among other advantages, the proteins that are produced using the present system result in a high mannose carbohydrate moiety. While often a disadvantage, this glycosylation pattern is useful for the targeting of certain proteins to macrophages. In preferred embodiments of the invention, a continuous fermentation process is employed to produce recombinant human glucocerebrosidase with high mannose content.

Other lysosomal storage disorders, whose associated lysosomal enzymes which may be suitable for expression in Pichia include Pompe's (alpha-glucosidase), Hurler's (alpha-L iduronidase), Fabry's (alpha-galactosidase), Hunters (MPS II) (iduronate sulfatase), Morquio Syndrome (MPS IVA)(galactosamine-6-sulfatase), and Maroteux-Lamy (MPS VI) (arylsulfatase B). Additional proteins that may be produced in accordance with the present invention include lysosomal acid lipase. In addition, any protein for which targeting to the macrophages is desired may be a suitable candidate for recombinant expression in Pichia, for example, by the continuous fermentation processes provided by the present invention.

Thus, in certain embodiments, the present invention comprises methods for the production of recombinant proteins with high-mannose glycosylation by expression in a Pichia cell expression system. The production process is preferably a continuous fermentation process.

In preferred embodiments, the process utilizes expression vectors comprising a constitutive promoter, such as the GAPDH promoter, operably linked to a coding DNA sequence. The preferred coding DNA sequences include any therapeutic protein for which activity and targeting are not adversely impacted by high-mannose glycosylation. In preferred embodiments, the coding DNA sequences comprise a sequence encoding a protein which is desired to be targeted to macrophages. In particular, preferred coding DNA sequences include those sequences encoding, glucocerebrosidase and acid sphingomyelinase, for the treatment of patients with Gaucher's Disease and Niemann-Pick Disease, respectively. Other preferred coding DNA sequences include those encoding alpha-glucosidase (Pompe's Disease), alpha-L iduronidase (Hurler's Disease), alpha-galactosidase (Fabry Disease), iduronate sulfatase (Hunters Disease (MPS II), galactosamine-6-sulfatase (MPS IVA), beta galactosidase (MPS IVB) and arylsulfatase B (MPS VI).

### BRIEF DESCRIPTION OF THE FIGURES

FIG. 1A. rh-Chitinase expression in methanol induced fed-batch culture with P. pastoris (host SMD 1168, His⁻, vector pPICZα). A 50% glycerol solution was fed during day one (0.3 ml/min). Subsequently, induction with methanol (0.12 ml/min) was initiated.

FIG. 1B. SDS-PAGE. Lane 2 and 3. rh-Chitinase standard containing full length and cleaved 37 kDa protein (both forms are active). Lane 4-7: supernatant from fed-batch culture, days 2-5.

FIG. 2A. Constitutive rh-Chitinase expression in fed-batch culture with P. pastoris (host SMD 1168, His⁻, vector pGAPZα). A 50% glycerol solution was fed (0.16 ml/min).

FIG. 2B. SDS-PAGE. Lane 2-5: supernatant from fed-batch culture, days 4-7.

FIG. 3. Constitutive rh-Chitinase expression in fed-batch culture with P. pastoris (host SMD 1168, His⁻, vector pGAPZα). A 50% glycerol solution was fed (0.16 ml/min) containing casamino acids (graph not shown). SDS-PAGE. Lane 2-5: supernatant from fed-batch culture, days 4-7.

FIG. 4A. Constitutive rh-Chitinase expression in continuous culture with P. pastoris (host SMD 1168, His⁻, vector pGAPZα). A 50% glycerol solution was fed (1.0 ml/min; 1.0 VVD).

FIG. 4B. SDS-PAGE. Lane 2-8: supernatant from continuous culture, days 2-8.

FIG. 5A. Constitutive rh-Chitinase expression in continuous culture with P. pastoris (host X33, vector pGAPZα). A 30% glucose solution was fed (1.2 ml/min; 1.2 VVD). Culture was run successfully for 30 days.

FIG. 5B. SDS-PAGE. Lane 2-5: supernatant from continuous culture, day 10-30.

FIG. 6. Glucose limited 15 L continuous culture of P. pastoris for rh-Chitinase production (D=0.04 h⁻¹; one volume exchange per day, sparged with conventional air).

FIG. 7. Comparison of 1.5 L continuous culture of P. pastoris (1.2 volume exchanges per day, sparged with molecular oxygen) with a cultivation at 15 L scale (one volume exchange per day, sparged with conventional air).

FIG. 8. k_{L}a, OTR, and impeller speed (N) vs. P/V_{L} in a 1,500 L STR (k_{L}a and OTR measured with the steady-state method, with: F/V_{L}=0.6 l/l min, p=1.01 bar, pO₂=0%, T=30° C.). In comparison, the operational set point of the 21 L CSTR in terms of k_{L}a and OTR (steady-state method) with: F/V_{L}=1.2 l/l min, p=1.61 bar, pO₂=35%, T=30° C.

FIG. 9. Model prediction for rh-Chitinase productivity (Q_{P}) and oxygen demand in terms of OUR and k_{L}a for further increased dilution rates (OUR based on p=1.61 bar and pO₂=35%) with: Y_{DCW/O2}=0.91, Y_{P/DCW}=1.4×10⁻³, and Y_{DCW/Glucose}=0.37.

FIG. 10. 1.5L continuous Pichia pastoris X33 culture for the expression of rh-GCR

FIG. 11. 15 L continuous Pichia pastoris SMD 1168 culture for the expression of rh-LAL

### DETAILED DESCRIPTION OF THE INVENTION

The continuous production processes of the present invention offer, in comparison to conventional fed-batch fermentation, advantages in terms of higher volumetric productivity, product quality, and product uniformity as the exposure of the product to proteolytic enzymes, oxidation or inactivation is significantly reduced. A continuous production process for rh-Chitinase using a constitutive P. pastoris expression system was recently developed by the inventors and compared very favorably in terms of cost effectiveness, development time, and effort to expression of rh-Chitinase in mouse C127 cells.

The P. pastoris production process has an extremely high oxygen demand due to the high cell densities obtained in the reactor. The oxygen demand is usually met by sparging with molecular oxygen (Chen (1); Chen (2); Siegel et al.) which presents a major economic and safety concern, especially at large-scale. Aerobic microbial high cell density cultures are usually run in stirred tank reactors (STR) and require the creation of a large air/water interface. The formation of the latter depends mainly on the realizable volume related power input into the reactor which is scale-dependent. The present invention provides methods for which air provides sufficient oxygen, and molecular oxygen is not needed. These methods have been scaled up to 15 L, and can potentially be further augmented for significantly larger scale processes, of up to 1000 L or more.

The present invention further provides processes for large-scale recombinant protein production using the constitutive P. pastoris expression system.

It is known that glycosylation of proteins expressed in Pichia is closer to that of mammalian cells compared to other yeasts and microorganisms. However, there are subtle differences. If glycosylation is critical to the function of the protein, e.g., activity and targeting, Pichia may not be suitable. However, many of the lysosomal enzymes, and in particular, glucocerebrosidase (Gaucher's Disease) and acid sphingomyelinase (Niemann-Pick Disease A & B), are particularly good candidates for treatment with recombinant protein produced in Pichia. This is because the majority of lysosomal storage enzymes naturally contain high-mannose oligosaccharides similar to Pichia derived proteins, and they have acidic optimal pH ranges which are found in lysosomes. For proteins that are targeted to macrophages by terminal mannoses, e.g., glucocerebrosidase and acid sphingomyelinase, the presence of mannose-6-phosphate may not be necessary. Pichia is an ideal expression system for expression of these proteins, because processing steps which may be necessary for trimming the carbohydrate chains produced by other expression systems, such as CHO, will not be required to expose mannose moieties.

Other lysosomal storage disorders, whose associated lysosomal enzymes which may be suitable for expression in Pichia include Pompe's (alpha-glucosidase), Hurler's (alpha-L iduronidase), Fabry's (alpha-galactosidase), Hunters (MPS II) (iduronate sulfatase), Morquio Syndrome (MPS IVA)(galactosamine-6-sulfatase), MPS IVB (beta-D-galactosidase), and Maroteux-Lamy (MPS VI)(arylsulfatase B). Other proteins that may be produced in accordance with the present invention include lysosomal acid lipase. There is evidence of other independent pathways, in addition to the mannose-6-phosphate pathway, that function in the transport of lysomal enzymes inside cells and of alternate mechanisms for the internalization of lysosomal enzymes by cell-surface receptors in addition to mannose-6-phosphate receptors (Scriver et al. 1995). In addition, any protein for which targeting to the macrophages is desired may be a suitable candidate for recombinant expression in Pichia, for example, by the continuous fermentation processes provided by the present invention.

Thus, in certain embodiments, the present invention comprises methods for the production of recombinant proteins with high-mannose glycosylation by expression in a Pichia cell expression system. The production process is preferably a continuous fermentation process.

In preferred embodiments, the process utilizes expression vectors comprising a constitutive promoter, such as the GAPDH promoter, operably linked to a coding DNA sequence. Other promoters of potential use in the present invention include constitutive promoters, such as the CMV promoter, the adenoviral major late promoter, and ubiquitin promoters, as well as inducible promoters, such as the alcohol oxidase promoter (Ellis et al., Mol. Cell. Biol. 9:1316-1323 (1985)); and the tetracycline inducible promoter system. The preferred coding DNA sequences include any therapeutic protein for which activity and targeting are not adversely impacted by high-mannose glycosylation. In preferred embodiments, the coding DNA sequences comprise a sequence encoding a protein which is desired to be targeted to macrophages. In particular, preferred coding DNA sequences include those sequences encoding, glucocerebrosidase and acid sphingomyelinase, for the treatment of patients with Gaucher's Disease and Niemann-Pick Disease, respectively. Other preferred coding DNA sequences include those encoding alpha-glucosidase (Pompe's Disease), alpha-L iduronidase (Hurler's Disease), alpha-galactosidase (Fabry's Disease), and iduronate sulfatase (Hunters Disease (MPS II), galactosamine-6-sulfatase (MPS IVA); beta-D-galactosidase (MPS IVB); and arylsulfatase B (MPS VI). In addition, a cDNA for any protein for which targeting to the macrophages is desired may be a suitable candidate for recombinant expression in Pichia, for example, by the continuous fermentation processes provided by the present invention.

Methods for the purification of recombinant human proteins are well-known, including methods for the production of recombinant human glucocerebrosidase (for Gaucher's Disease); sphingomyelinase (for Niemann-Pick Disease), alpha-galactosidase (for Fabry Disease); alpha-glucosidase (for Pompe's Disease); alpha-L iduronidase (for Hurler's Syndrome); iduronate sulfatase (for Hunter's Syndrome); galactosamine-6-sulfatase (for MPS IVA); beta-D-galactosidase (for MPS IVB); and arylsulfatase B (for MPS VI). See, for example, Scriver et al., eds., The Metabolic and Molecular Bases of Inherited Diseases, Vol. II., 7^{th} ed. (McGraw-Hill, NY; 1995), the disclosure of which is hereby incorporated herein by reference.

While the invention is exemplified with respect to the production of specific proteins, these examples are not to be interpreted as limiting the invention in any manner. As described above, and as will be clear to those skilled in the art from reading the specification, the methods of the present invention are useful for production of numerous other recombinant proteins, including the lysosomal enzymes described above. Many modifications and variations of the methods and materials used in the present description will also be apparent to those skilled in the art. Such modifications and variations fall within the scope of the invention.

The entire disclosures of all of the publications and references cited in this specification are hereby incorporated herein by reference.

### EXAMPLES

### Example 1

### Cloning and Selection of the Human Chitinase (hChitinase) Gene in P. pastoris

a. Vector Construction

The hChitinase cDNA was received from Johannes Aerts, University of Amsterdam, NL (WO 9640940) and used as a template for all PCR reactions. The coding region of hChitinase without the secretion signal peptide and containing Eco RI sites at the 5′ and 3′ ends was generated by PCR and inserted into Eco RI linearized pPICZα and pGAPZα, which contain the S. cerevisiae α-factor secretion signal. The coding region of hChitinase with it's secretion signal peptide and Eco RI sites at the 5′ and 3′ ends was generated by PCR and inserted into Eco RI linearized pGAPZα. All vectors were obtained from Invitrogen(San Diego, Calif.).

b. Transformation

P. pastoris cells were made competent and transformed by electroporation as previously described (Becker et al., 1991) with slight modifications. P. pastoris strains X33 and SMD1168 (Invitrogen) were grown to OD₆₀₀ of 0.5-0.8. in a 50 ml culture, pelleted and resuspended in 10 ml ice-cold 100 mM Tris, 10 mM EDTA buffer with 200 mM DTT (Sigma), and incubated for 15 minutes at 30° C. with shaking at 100 rpm. Cells were then washed 2× with ice-cold sterile water and 1× with 1 M sorbitol (Invitrogen) and resuspended in 100 μl 1 M sorbitol to a final volume of ≈200 μl. 80 μl competent cells were electroporated with 2-6 μg DNA in 0.2 cm cuvettes at 1500 V, 25 μF and 200 Ω using a BioRad Gene Pulser with pulse controller. Immediately after pulsing, 1 ml of ice-cold sorbitol was added to the cuvette. Cells were allowed to recover overnight at room temperature, then plated (20-100 μl cells per plate) directly on YPD (Yeast Extract, Potato, Dextrose medium, Invitrogen) agar containing differing amounts of zeocin (Invitrogen) for selection. Plates were incubated at 30° C. Resistant colonies appeared after 2 days on 0.1-0.5 mg/ml zeocin and after 3-4 days on 1-2 mg/ml zeocin.

c. Selection of High Producers

Several hundred clones that survived higher titers (0.5-2 mg/ml) of zeocin were screened in test tubes as follows. A single colony was inoculated into 5 ml of YPD in 50 ml conical centrifuge tubes and incubated for 24 hours at 30° C. with shaking at 250 rpm. Cell density was measured by OD₆₀, and a fresh 5 ml YPD was inoculated with 2.5×10⁶ cells and incubated as above. This process was repeated as necessary until cells from each clone being analyzed were synchronized in growth. Typically two or three passages were sufficient. Once synchronized, cells were grown for 60 hours as above. Aliquots of culture (50 μl) were aseptically removed at 24, 48 and 60 hours and conditioned medium was harvested and analyzed by the pNP(Sigma) activity assay as described below to identify top producers.

d. 1.5-L Fed-Batch Fermentation

A shake flask containing 100 ml of YPD medium was inoculated with one vial (˜1 ml, OD₆₀₀=25) containing a recombinant P. pastoris cell line. The flask was incubated at 30° C. and 220 min⁻¹ for 16-24 hours, until the cell density reached OD₆₀₀>15. YPD medium (pH=6.0) used in shake flask cultivation consisted of (per liter deionized water): D-glucose 20 g, soy peptone 20 g, yeast extract 10 g, yeast nitrogen base (w/o amino acids) 13.4 g, KH₂PO₄ 11.8 g, K₂HPO₄ 2.3 g, D-biotin 0.4 mg.

The cells from this flask were used to inoculate a 3.0-L fermenter (Applikon, Foster City, Calif.) with a 1.5-L working volume at a density of 1.0 to 2.0 OD₆₀₀ units. The fermenter contained Basal Salts Medium plus 2 g/L Histidine for His⁻ strains. Basal Salts Medium used for fermenter batch cultivation contained (per liter deionized water): Glucose 40 g, H₃PO₄ (85%) 26.7 ml, K₂SO₄ 18.2 g, MgSO₄.7H₂O 14.9 g, KOH 4.13 g, CaSO₄.2H₂O 0.93 g, D-biotin 0.87 mg, trace salts solution 4.35 ml; (trace salts solution(per liter deionized water): Fe₂(SO₄).7H₂O 65 g, ZnSO₄ 42.19 g, CuSO₄.5H₂O 6 g, MnSO₄.H₂O 3 g, CoCl₂.6H₂O 0.5 g, Na₂MoO₄.2H₂O 0.2 g, NaI 0.08 g, H₃BO₃ 0.02 g).

The cells were grown batchwise until the initial glucose was depleted (˜24 hours) and the wet cell weight (WCW) was ˜80-100 g/L. When the initial glucose was depleted as indicated by a dissolved oxygen (pO₂) spike, fed-batch fermentation was initiated by starting the fed-batch medium at a rate of 0.13-0.20 mL/L initial medium volume. The fed-batch medium consisted of (per liter deionized water): D-glucose 500 g, D-biotin 2.4 mg, trace salts solution 12 mL, and casamino acids 10 g (in circumstances when such use is mentioned in present description of production of specified proteins).

Fed-batch fermentation was continued until activity had plateaued (˜5-7 days). Samples were taken daily for WCW and cell density by OD₆₀₀. Supernatant was obtained by centrifugation at 4-6,000 g for 25 min. at 4° C. and stored at −20° C. until assayed.

e. 1.5-L Continuous Fermentation

After the fed-batch fermentation had been established (see above), and allowed to continue for approximately 24 hours (WCW ˜200-220 g/L), continuous fermentation was initiated at a rate of 0.7-0.8 Volumes/Working Volume/Day (VVD). The continuous feed medium (pH=1.3) contained (per liter deionized water): D-glucose 300 g, H₃PO₄ (85%) 13.35 ml, K₂SO₄ 9.1 g, MgSO₄.7H₂O 7.45 g, KOH 2.07 g, CaSO₄.2H₂O 0.47 g, D-biotin 0.87 mg, and trace salts solution 4.35 ml.

After ˜24 hours of continuous culture, the continuous flow rate was increased to ˜1.0-1.2 VVD, or ˜0.7-0.85 ml/min/L working volume. Flow rate was maintained in this range for the duration of the run. Samples were taken daily for WCW and cell density by OD₆₀₀. Supernatant was obtained and stored at −20° C. for recombinant protein concentration measurements.

The continuous outflow of culture was harvested daily and supernatant was obtained by centrifugation at 4-6,000 g for 25 min. at 4° C. and stored at −20° C. until assayed.

f. rhChitinase Activity Assay

Crude supernatant (1:100 to 1:1000) or pNP standard (Sigma) (0-20 nM/well) were diluted in assay buffer pH 5.2. 100 μl of standards and diluted crude supernatant were placed into duplicate wells in a 96 well microtiter plate. One hundred μl of substrate, 0.25 mg/ml pNP-β-N,N′-diacetylchitobiose (Sigma), was then added to each well and the plate incubated at 37° C. with shaking at 50 rpm. After 2 hours, 50 μl of 1.0 N NaOH was added to each well and the absorbance at 405 nm against 650 nm(reference) was read using a microtiter plate reader.

Activity was determined using a pNP standard curve. A specific activity(determined using purified material at Genzyme) of 1.67 U/mg was used to convert activity units [U/ml] to protein units [mg/ml].

g. SDS Page and Gel Staining

About 10 μl supernatant of each sample (2-4 μg protein) was mixed with 20 μl 5×SDS non-reducing sample loading buffer (BioRad, CA) and 30 μl was subjected to electrophoresis on 4-20% Tris Glycine acrylamide mini-gels (Ready Gel, BioRad, CA) in tris-glycine-SDS running buffer (BioRad, CA). Gels were stained with Coomassie-blue staining reagent (BioRad, CA) for about one hour, then destained with 40% methanol/10% acetic acid for one hour.

h. Results and Discussion

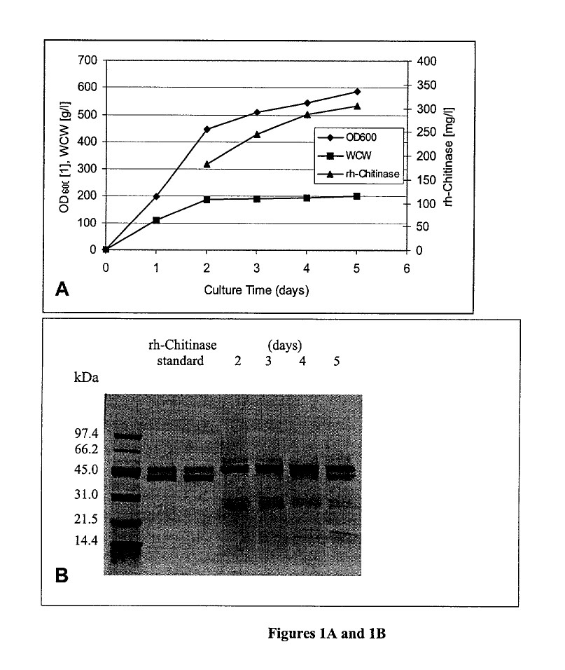

i. Fed Batch Production of rh-Chitinase by a Methanol-Inducible clone (pPICZα-SMD 1168) Cell yield measured by WCW of the culture plateaued after 2 days at 200 g/L while activity increased slowly through day 5. Final rh-Chitinase concentration in the culture broth reached a moderate level of 300 mg/L (FIG. 1a). However, degradation of the rh-Chitinase was evident on day 4 when time samples were analyzed on SDS page gel (FIG. 1b). Two distinct bands can be seen in samples collected on the 5^{th} day. These data may explain why the rh-Chitinase activity only increased slowly with time under fed-batch mode.

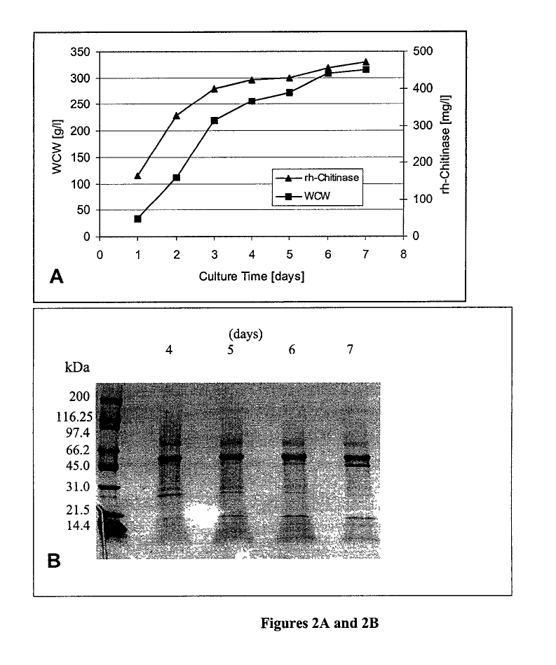

ii. Fed Batch Production of rh-Chitinase by a Constitutive Clone (pGAPZα-SMD 1168) When pGAPZα-SMD 1168 was grown under fed-batch conditions, cell yield reached 330 g/L WCW and a rh-Chitinase concentration of 450 mg/L was attained (FIG. 2a). A similar degradation pattern was seen with the recombinant protein. A second lower MW band began to appear after 6 days and the band became more prominent on day 7, suggesting proteolytic degradation (FIG. 2b).

iii. Protection of Enzyme from Proteolytic Degradation by Casamino Acids Supplementation

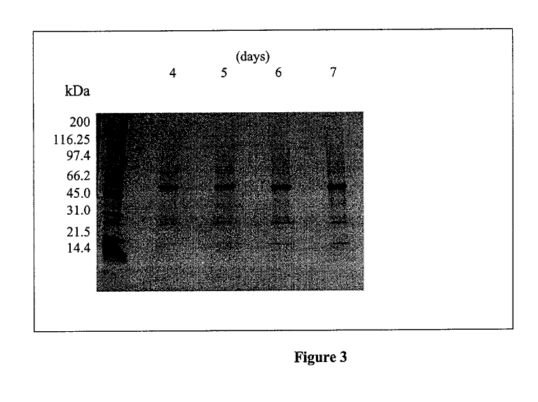

Casamino acids have been shown to protect proteins from proteolytic degradation when added to cultures. They were included in the fed-batch feed medium and samples (4,5,6 & 7days) were collected and analyzed by SDS PAGE. A tight band at around 50 kDa in each one of the samples analyzed suggests intact rh-Chitinase(FIG. 3). This can be compared to samples from a fed batch fermentation without casimino acids which showed a low MW band on day 6(FIG. 2b). These data suggests that rh-Chitinase was most likely degraded by proteolytic enzymes under fed batch conditions and that rh-Chitinase can be stabilized by addition of casimino acids.

Athough casimino acids appeared to be effective in preventing proteolytic degradation of rh-Chitinase in the fermentation broth, this method may not be ideal for production because of the animal origin of such casimino acids.

iv. Stabilization of rh-Chitinase by Continuous Fermentation

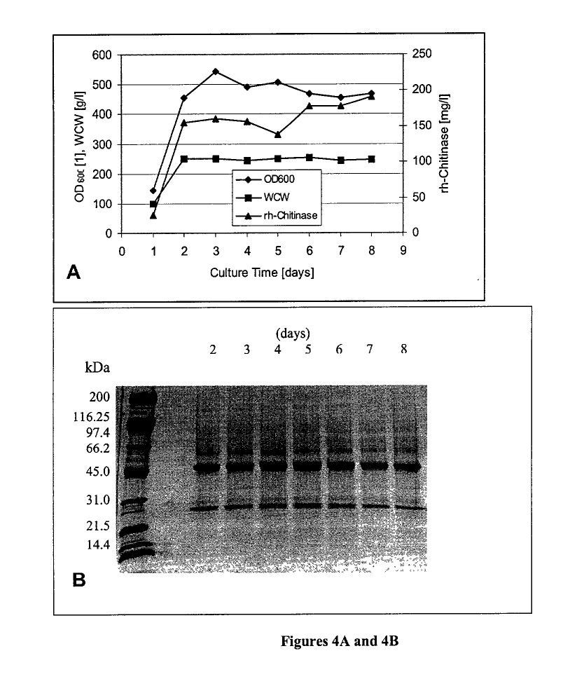

FIG. 4a shows rh-Chitinase and growth data of the constitutive clone (pGAPZα-SMD 1168) in a continuous mode. Medium was exchanged at a rate of 1.0 VVD. The culture reached steady-state on day 2 of continuous mode and rh-Chitinase was produced at a volumetric productivity of 180 mg/L/d. The fermentation was continued for 26 days and samples from day 2 through 8 were analyzed on SDS PAGE. The gel shows a single rh-Chitinase band (˜50 kDa) in all samples (FIG. 4b) indicating that continuous fermentation can prevent degradation of rh-Chitinase for at least up to 8 days. It appears that little or no proteolytic enzyme(s) is produced and released by the culture into the medium under continuous cultivation. It is also possible that when the protein is harvested continuously, it is exposed to less concentrated proteolytic enzymes for a much shorter time period compared to rh-Chitinase production under fed batch conditions. SDS PAGE of samples after day 8 were not performed because the onset of protease typically occurred much before day 8.

v. pGAPZα-X33 Clone

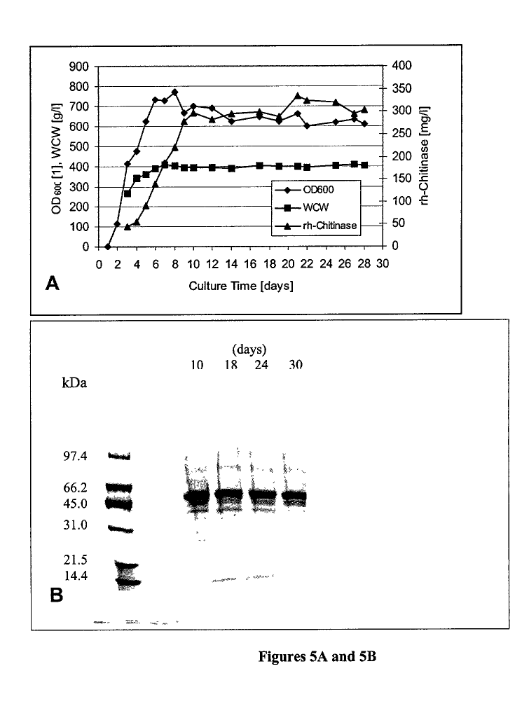

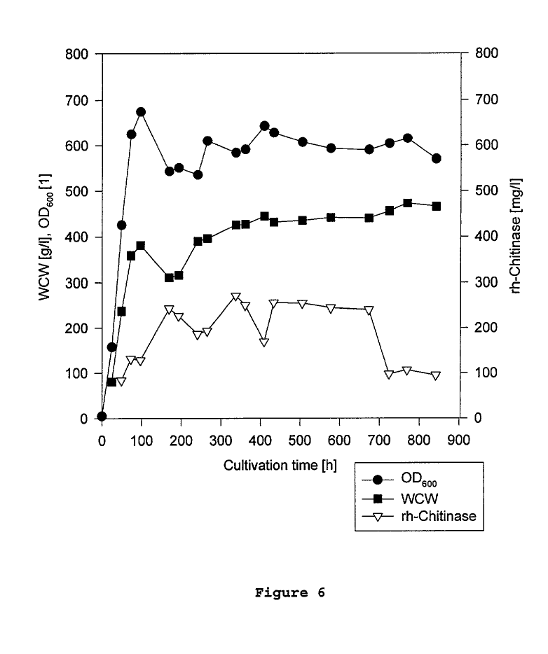

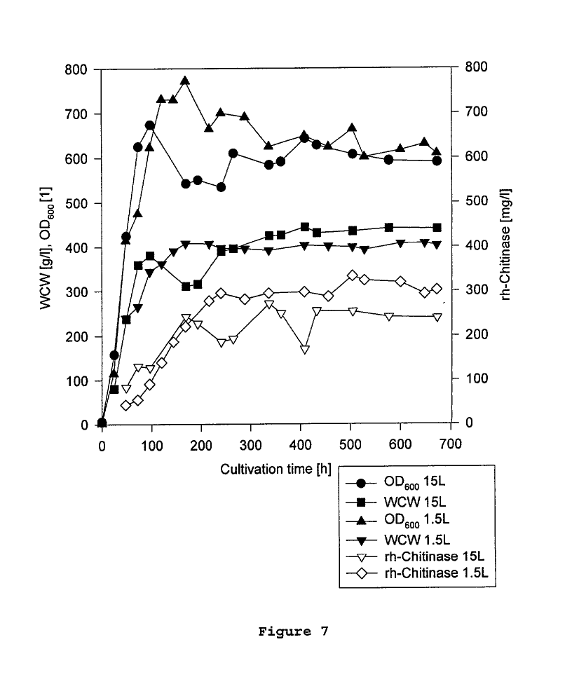

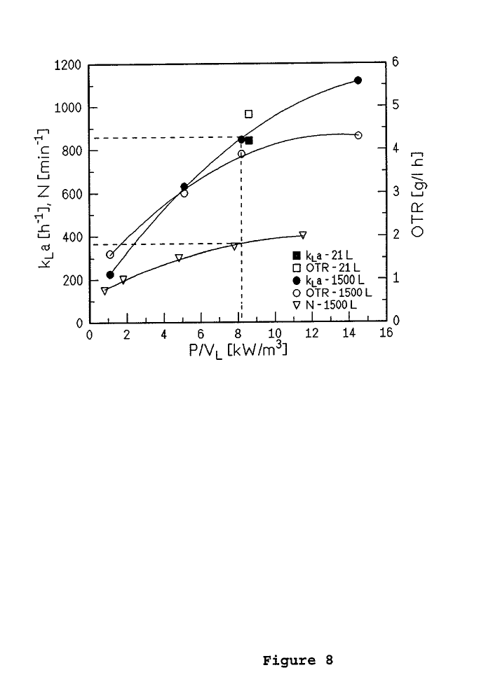

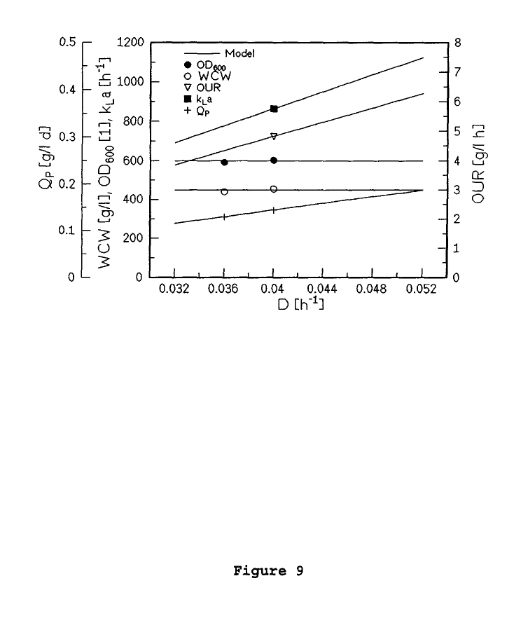

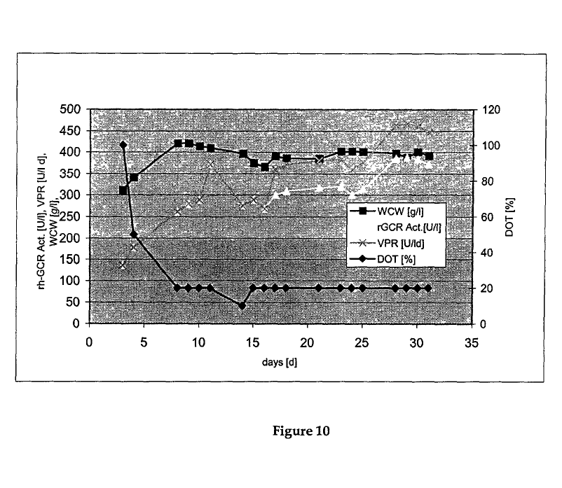

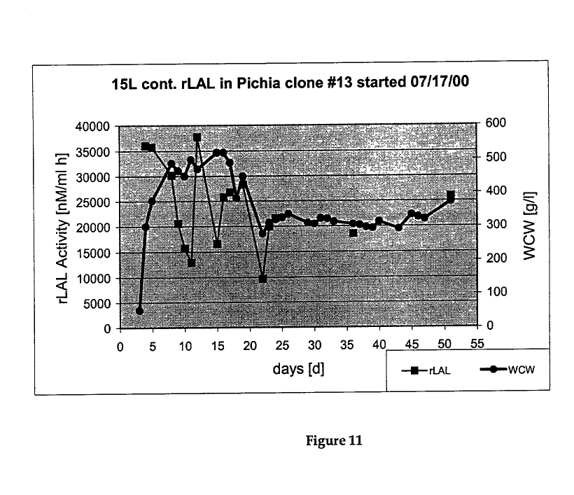

The highest producing clone was created when the X33 host was used for the transformation. This clone was grown in the continuous mode with an initial dilution rate of 0.8 VVD. The feeding rate was ramped up slowly to 1.2 VVD on day 6 (FIG. 5a). rh-Chitinase concentration increased steadily from 50 mg/L to 300 mg/L within a period of 8 days. Cell yield plateaued on day 5 (˜400 g/L WCW) and rh-Chitinase concentration plateaued on day 9 (˜300 mg/L). The culture was continuously fed with 30% glucose feed medium, as discussed in the present description of the invention, at a rate of 1.2 VVD for an additional 24 days. The cell yield and rh-Chitinase volumetric productivity remained steady at 400 g/L WCW and 360 mg/Ld, respectively. As far as we know, this is the first report describing a P. pastoris high cell density fermentation continuing for 30 days. The culture showed no signs of decline, both in cell and product yields at run termination. SDS PAGE analysis of samples indicated that the product was not degraded even on the 30^{th} day of the fermentation (FIG. 5b). We have since cloned two other therapeutic proteins (one antiangiogenesis protein & one lysosomal enzyme) with the GAP promoter and produced them using continuous conditions. Both recombinant proteins, which normally were digested by proteases under fed-batch conditions, were not degraded.

### CONCLUSIONS

A process for the cultivation of P. pastoris in continuous fermentation using the constitutive GAP promoter for the production of recombinant proteins has been developed. To our knowledge this is the first use of a continuous high cell density fermentation process employing the constitutive expression vector (pGAPZα) in P. pastoris. Also, the constitutive expression system allows for the safe handling of the P. pastoris production system, especially in large scale, avoiding the use of methanol, which is flammable. This would greatly reduce the hazard and costs involved with large scale production of recombinant therapeutic proteins in P. pastoris by alleviating the need for explosion proof GMP facilities.

This continuous system provides not only for greatly enhanced production of recombinant proteins and reduction of down-time associated with fermentor turn around (approximately 6 fold higher productivity than fed-batch fermentation) but also for the production of intact proteins that are usually degraded in a fed-batch mode. This may be due to the continual separation of sensitive proteins from the culture broth. It is believed that this continuous Pichia expression system, employing the GAP promoter, is applicable to a wide range of proteins which previously could not be produced in methylotrophic Pichia due to proteolytic degradation and/or economic reasons.

### EXAMPLE 2

### Scale-Up of a High Cell Density Continuous Culture with Pichia pastoris X-33 for the Constitutive Expression of rh-Chitinase

List of symbols

| | | |
|---|---|---|
| c_{DCW} | g/l | dry cell weight concentration |
| c_{P} | g/l | product concentration in the supernatant |
| c_{02, L} | g/l | dissolved oxygen concentration |
| c*_{02, L} | g/l | oxygen solubility |
| CER | g/l h | carbon dioxide evolution rate |
| d | m | impeller diameter |
| D | h⁻¹ | dilution rate |
| F^{N} | l/min | air flow rate (under standard conditions) |
| F/V_{L} | l/1 min | volume related aeration rate |
| He | g/l bar | Henry constant |
| k_{L}a | h⁻¹ | volume related oxygen transfer coefficient |
| MW | g/mol | molecular weight |
| N | min⁻¹ | impeller speed |
| OTR | g/l h | oxygen transfer rate |
| OUR | g/l h | oxygen uptake rate |
| p | bar | pressure |
| p^{N} | bar | pressure (under standard conditions) |
| pO₂ | % | oxygen partial pressure in the liquid phase |
| P/V_{L} | kW/m³ | volume related power input |
| qO₂ | h⁻¹ | specific oxygen uptake rate (g O₂/g DCW h) |
| Qₚ | g/l d | volumetric productivity |
| r | % | percentage of solids in fluid |
| R | bar 1/K mol | gas constant |
| RQ | l | respiratory quotient |
| T^{N} | K | temperature (under standard conditions) |
| U | μM/min | enzyme activity |
| V_{L} | l | reactor liquid volume |
| X_{02, in} | l | mol fraction of O₂ in inlet gas |
| X_{C02,in} | l | mol fraction of CO₂ in inlet gas |
| X_{02, out} | l | mol fraction of O₂ in exhaust gas |
| X_{C02, out} | l | mol fraction of CO₂ in exhaust gas |
| Y_{DCW/02} | l | yield coefficient (biomass formed/oxygen |
| | | consumed) |
| Y_{P/DCW} | l | yield coefficient (rh-Chitinase produced/bio- |
| | | mass formed) |

Greek symbols

| | | |
|---|---|---|
| η | Pas | shear viscosity |
| μ | h⁻¹ | specific growth rate |

Introduction:

The feasibility of large-scale production of recombinant human chitinase using a constitutive Pichia pastoris expression system was demonstrated in a 21 L continuous stirred tank reactor (CSTR). A steady-state recombinant protein concentration in the supernatant of 250 mg/l was sustained for one month at a dilution rate of D=0.04 h⁻¹ (equivalent to one volume exchange per day), enabling a volumetric productivity of 144 mg/l d (240 U/l d). The steady-state dry cell weight concentration in this high cell density culture reached 110 g/l. Considering safety and economical aspects, all large-scale cultivations were conducted without molecular oxygen supplementation. Conventional air sparging was used instead. The oxygen demand of the process was determined by off-gas analysis (OUR=4.8 g O₂ l⁻¹ h⁻¹ with k_{L}a=846 h⁻¹) and evaluated with regard to further reactor scale-up.

A. Microorganism and Conditions of Cultivation

Cultivations were carried out with the yeast Pichia pastoris X-33, wild type strain His⁺ (Invitrogen, San Diego, Calif.) using a pGapZα vector for constitutive expression of rh-Chitinase. Vials with 1 ml of frozen working stock of recombinant P. pastoris were stored at −80° C. and used as inoculum for 2 L shake flask cultivations with 500 ml YPD medium. Two 2 L shake flasks with 500 ml YPD medium were inoculated and incubated for ˜24 h at 30° C. on a orbital shaker at 220 min⁻¹ until cell density reached OD₆₀₀>50. YPD medium (pH=6.0) used in shake flask cultivations consisted of (per liter deionized water): D-glucose 20 g, soy peptone (Type IV, Sigma, MO) 20 g, yeast extract (HyYest 444, Quest, Ill.) 10 g, yeast nitrogen base (w/o amino acids) (Difco, Mich.) 13.4 g, KH₂PO₄ 11.8 g, K₂HPO₄ 2.3 g, D-biotin 0.4 mg. Each bioreactor cultivation was seeded with the contents of two 2L shake flask cultures, equivalent to 6.6% v/v culture suspension.

Bioreactor cultivations were performed in a 21 L stirred tank reactor (STR) with 15 L working volume (CF 3000, Chemap AG, Switzerland) and a height/diameter ratio of 2.0. The reactor had four baffles and three Rushton impellers (d=0.075 m) installed on the shaft at ¼, ½, and ¾ of the liquid level. Cultivation parameters were set to T=30° C., pH=5.0 [adjusted with NH₄OH (30%) and H₃PO₄ (40%)], N=600-1,000 min⁻¹, F/V=1.0-2.0 l/l min, p=1.01-1.61 bar, and pO₂≧20%. The medium used for bioreactor batch cultivations contained (per liter deionized water): D-glucose 40 g; H₃PO₄ (85%) 26.7 ml; K₂SO₄ 18.2 g; MgSO₄.7H₂O 14.9 g; KOH 4.13 g; CaSO₄.2H₂O 0.93 g; D-biotin 0.87 mg; and trace salts solution 4.35 ml. Trace salts solution consisted of (per liter deionized water): Fe₂(SO₄).7H₂O 65 g; ZnSO₄ 42.19 g; CuSO₄.5H₂O 6 g; MnSO₄.H₂O 3 g; CoCl₂.6H₂O 0.5 g; Na₂MoO₄.2H₂O 0.2 g; NaI 0.08 g; and H₃BO₃ 0.02 g. After ˜24 h cultivation time, the OD₆₀₀ reached 150 and the initial glucose was depleted. 4.5 L fed-batch medium in a 5 L bottle were fed into the reactor at a feed rate of 3 m/min. The fed-batch medium (pH 7.0) consisted of (per liter deionized water): D-glucose 500 g and D-biotin 2.4 mg. After 24 h of fed-batch mode, an OD₆₀₀ of 450 was attained and the reactor was switched to continuous mode at a medium feed rate of 7 ml/min, which was increased to <11 ml/min after 24 h. The feed medium (pH=1.3) used for continuous cultivation contained (per liter deionized water): D-glucose 300 g, H₃PO₄ (85%) 13.35 ml, K₂SO₄ 9.1 g, MgSO₄.7H₂O 7.45 g, KOH 2.07 g, CaSO₄.2H₂O 0.47 g, D-biotin 0.87 mg, and trace salts solution 4.35 ml. Glucose limitation of the culture was maintained and monitored during fed-batch and continuous mode of operation. Steady-state condition was usually reached after 3-5 volume exchanges. The media used in 2 L shake flasks, 5 L bottles, and a 21 L bioreactor was sterilized for 30 min at 121° C. The feed medium used for continuous production was filter-sterilized into 200 L plastic bags. The harvest was continuously pumped into a sterile 130 L plastic bag which was placed in a 150 L chilled vessel (4° C.). Twice a week, the harvest was filled into 1 L plastic bottles and centrifuged (Sorvall RC3C, DuPont Corp.) at 4,500 min⁻¹ (5,900 g) for 45 min at 4° C. The supernatant was concentrated by cross flow filtration (Pellicon, Millipore; membrane cut-off: 10 kDa) and frozen at −80° C. for further protein purification.

B. Analysis

OD was measured at 660 nm (spectrophotometer model 8452A, Hewlett Packard). Wet cell weight (WCW) was determined gravimetrically after centrifugation (Allegra 21 R, Beckman) of 50 ml cell suspension at 6,500 min⁻¹ (4,400 g) for 25 min at 4° C. Dry cell weight (DCW) estimation included washing of the pellet and drying of the sample at 40° C. for 72 h. The D-glucose concentration was measured with an Accu-Chek glucose analyzer (Boehringer Mannheim, Germany) for confirmation of glucose limitation in the CSTR. Exhaust analysis of carbon dioxide, oxygen, nitrogen, and water was measured by mass spectrometer (MGA 1600, Perkin-Elmer, USA). Carbon dioxide evolution rate (CER), oxygen uptake rate (OUR), and respiratory quotient (RQ) were evaluated from the gas phase material balance. Bioreactor and cultivation parameters such as N, T, pH, and pO₂ were documented via chart recorder (Yokogawa). Power input was determined by measurement of electrical voltage and current at the armature of the motor. Friction losses were subtracted.

C. rh-Chitinase Activity Assay

rh-Chitinase activity in the supernatant was determined via enzymatic essay. Crude supernatant or pNP (p-Nitrophenol) standard were diluted in assay buffer (0.02% NaAzide, pH 5.2). 100 μl of standards and diluted crude supernatant were placed into duplicate wells in a 96 well microtiter plate. 100 μl of substrate (0.25 mg/ml pNP-β-N,N′-diacetylchitobiose) was then added to each well and the plate was incubated for two hours in the dark at 37° C. with shaking at 50 rpm. After two hours, 50 μl of 1.0 N NaOH was added to each well and the absorbance at 405 nm to 650 nm reference was measured using a microtiter plate reader (340 ATTC, SLT, Salzburg). Activity was determined via pNP standard curve. A specific activity of 1.67 U/mg was used to convert activity units [U/ml] to protein units [mg/ml] (determined using purified material at Genzyme).

D. Calculation of rh-Chitinase Productivity

A change in the biomass concentration in the CSTR can be described by:

dc_{DCW}/dt=μc_{DCW}−Dc_{DCW} (1)

Steady-state condition of the continuous culture was reached when dc_{DCW}/dt=0 and consequently μ=D. With c_{P} as the steady-state rh-Chitinase concentration in the supernatant and r as the percentage of solids in the reactor fluid, the rh-Chitinase productivity can be calculated as:

Q_{P}=c_{P}D[1−(r/100] (2)

The oxygen consumption utilized for biomass formation can be described as:

dc_{DCW}/dt=OUR Y_{DCW/O2} (3)

rh-Chitinase production is assumed to be associated with growth:

dc_{P}/dt=Y_{P/DCW}·dc_{DCW}/dt (4)

E. Calculation of Oxygen Demand of Process

The oxygen demand of the process in terms of k_{L}a and oxygen transfer rate (OTR) can be estimated as follows. A change in the dissolved oxygen concentration in the reactor can be expressed as:

dc_{O2,L}/dt=OTR−OUR=k_{L}a (c^{*}_{O2,L}−c_{O2,L})−OUR (5)

In a small time interval, dc_{O2,L}/dt=0 and consequently

OTR=k_{L}a (c^{*}_{O2,L}−c_{O2,L})=OUR (6)

The oxygen uptake rate (OUR) can be determined via oxygen mass balance derived from exhaust analysis:

$$OUR = \frac{{F}^{N} {p}^{N} {x}_{O2 , i n} M {W}_{O2}}{{V}_{L} {R}^{N} {T}^{N}} [ 1 - \frac{{x}_{O2 , out} ( 1 - {x}_{O2 , i n} - {x}_{CO2 , i n} )}{{x}_{O2 , i n} ( 1 - {x}_{O2 , out} - {x}_{CO2 , out} )} ] \qquad ( 7 )$$

Assuming an ideally mixed gas phase in the reactor, with

c^{*}_{O2,L}=x_{O2,out}p/He (8)

and c_{O2,L}=c^{*}_{O2,L}pO₂/100 (9)

the k_{L}a can be calculated (steady-state method).

A correlation for k_{L}a using the parameters power input per volume (P/V_{L}), volume related aeration rate (F/V_{L}), and viscosity (η) can be described as:

k_{L}a=c (P/V_{L})^{a} (F/V_{L})^{b} (η)^{d} (10)

P/V_{L=}idem. is a common scale-up strategy to secure an equal oxygen supply in the larger reactor (to attain k_{L}a=idem. and OTR=idem.). Assuming geometrical similarity of the larger reactor, turbulent flow, F/V_{L}=idem., and η=idem.:

P˜N³ d⁵ and P/V_{L}˜N³d² (11)

With P/V_{L}=idem.: N_{large}/Nₛₘₐₗₗ=(dₛₘₐₗₗ/d_{large})^{⅔} (12)

The necessary impeller speed to secure an equal oxygen supply in the larger reactor can be calculated as shown in Eq. (13):

N_{large}=Nₛₘₐₗₗ(dₛₘₐₗₗ/d_{large})^{⅔} (13)

### Example 3

### rh-LAL (Lysosomal Acid Lipase)

rh-LAL was expressed in a 15L continuous culture with Pichia pastoris SMD 1168 (auxotrophic: His⁻) using the constitutive GAPDH promoter. Under steady-state conditions and a volumetric turnover rate of 1.0 VVD, an average LAL activity in the supernatant of 25,000 [nM/ml h] was attained with an average WCW of 350 [g/l]. All culture conditions were identical compared to 15L rh-Chitinase production (e.g. pH at 5.0, DOT controlled at 30% by air sparging).

It was found that a 75% reduction in TMS (compared to ‘Pichia Fermentation Guidelines’ by Invitrogen, CA) combined with a lower VVD of 1.0 could suppress protease activity in the medium and protect the LAL-product.

LAL activity was measured via fluorometric assay (similar assay published by: Grabowski, J Biol Chem, 270, 27766 (1995)).

### Example 4

### Pichia Expression of rh-GCR (Glucocerebrosidase)

rh-GCR was expressed in a 1.5 L continuous culture with Pichia pastoris X33 (prototrophic strain) using the constitutive GAP promoter. Under steady-state conditions, a maximum volumetric productivity (VPR) of 466 [U/L day] was attained at a volumetric turnover rate of 1.2 [volume/volume day] (VVD). GCR activity in the supernatant was 388 [U/L] and the wet cell weight (WCW) was 388 μg/l]. All culture conditions were identical compared to 1.5 L rh-Chitinase production (e.g. pH at 5.0, dissolved oxygen tension (DOT) controlled at 30% by oxygen sparging).

A 23% increase in VPR (from 380 [U/L day] to 466 [U/L day]) was achieved when the trace metal solution (TMS) in the medium was reduced by 50% on day 28 (compared to rh-Chitinase process which was based on the ‘Pichia Fermentation Guidelines’ by Invitrogen, CA). It was assumed that trace metals may catalyze GCR degradation. The GCR-CHO process adds DTT to the medium to protect the GCR from oxidation. The GCR-Pichia process did not need the addition of DTT, as GCR is not being oxidized at pH 5.0.

GCR activity can be tested according to conventional assays, such as the PNP-Beta-D-Glucopyranoside activity assay.

We claim:

1. A method for the production of recombinant proteins with high-mannose carbohydrate structure, comprising continuously culturing cells of Pichia pastoris, which cells comprise a DNA molecule which encodes a protein of interest, under conditions suitable for the expression of said DNA molecule.

2. The method of claim 1, wherein the recombinant proteins are human lysosomal enzymes selected from the group consisting of lysosomal acid lipase, alpha glucosidase, alpha-L idronidase, alpha galactosidase, iduronate sulfatase, galactosamine-6-sulfatase, beta galactosidase, and arylsulfatase B.

3. The method of claim 1, wherein the DNA molecule comprises a promoter operatively linked to a DNA coding sequence.

4. The method of claim 3, wherein the constitutive promoter is the GAPDH promoter.

5. The method of claim 4, wherein the cells are cultured without the addition of molecular oxygen.

6. A method for the production of recombinant glucocerebrosidase with high-mannose carbohydrate structure, comprising culturing cells of Pichia pastoris which cells comprise a DNA molecule which encodes glucocerebrosidase, under conditions suitable for the expression of said DNA molecule.

7. The method of claim 6, wherein the DNA molecule comprises a constitutive promoter operatively linked to a coding sequence for glucocerebrosidase.

8. The method of claim 6, wherein the cells are continuously cultured without the addition of molecular oxygen.

9. A method for purification of recombinant human glucocerebrosidase with high-mannose carbohydrate structure, comprising culturing cells of Pichia pastoris which cells comprise a DNA molecule which encodes glucocerebrosidase, under conditions suitable for the expression of said DNA molecule to produce recombinant human glucocerebrosidase in a cell culture, and purifying said produce recombinant human glucocerebrosidase from said cell culture.

10. The method of claim 9, wherein the DNA molecule comprises a constitutive promoter operatively linked to a coding sequence for glucocerebrosidase.

11. The method of claim 9, wherein the cells are continuously cultured without the addition of molecular oxygen.

12. A method for the production of recombinant sphingomyelinase with high-mannose carbohydrate structure, comprising culturing cells of Pichia pastoris which cells comprise a DNA molecule which encodes sphingomyelinase, under conditions suitable for the expression of said DNA molecule.

13. The method of claim 12, wherein the DNA molecule comprises a constitutive promoter operatively linked to a coding sequence for sphingomyelinase.

14. The method of claim 12, wherein the cells are continuously cultured without the addition of molecular oxygen.

15. A method for purification of recombinant human sphingomyelinase with high-mannose carbohydrate structure, comprising culturing cells of Pichia pastoris which cells comprise a DNA molecule which encodes sphingomyelinase, under conditions suitable for the expression of said DNA molecule to produce recombinant human sphingomyelinase in a cell culture, and purifying said produce recombinant human sphingomyelinase from the cell culture.

16. The method of claim 15, wherein the DNA molecule comprises a constitutive promoter operatively linked to a coding sequence for sphingomyelinase.

17. The method of claim 15, wherein the cells are continuously cultured without the addition of molecular oxygen.
